(19) United States
(12) Patent Application Publication
(10) Pub. No.: US 2004/0138639 A1
(43) Pub. Date: Jul. 15, 2004

(54) Disposable wearing article

(76) Inventors: Kyoko Ito, Kagawa-ken (JP); Junko Sato, Kagawa-ken (JP); Toru Sasaki, Kagawa-ken (JP)

Correspondence Address: LOWE HAUPTMAN GILMAN AND BERNER, LLP, 1700 DIAGONAL ROAD, SUITE 300 /310, ALEXANDRIA, VA, 22314, US

(21) Appl. No.: 10/685,695

(22) Filed: Oct. 16, 2003

(30) Foreign Application Priority Data
Oct. 17, 2002 (JP) 2002-303498
Oct. 1, 2003 (JP) 2003-343595

(51) Int. Cl. A61F 13/15

(52) U.S. Cl. 604/389

(57) ABSTRACT

Each of tape fasteners attached to a wearing article and maintained folded in a Z- or an inverted Z-shape. The Z- or to the inverted Z-shape is defined by a first tape segment having a free end zone, a second tape segment fixed to a surface of the wearing article to contact with the wearer's clothes and a third tape segment interposed between the first and second tape segments. An inner end zone of the third tape segment is connected to an inner end zone of the second tape segment and extends inwardly of the wearing article beyond the inner end zone to be releasably attached to the surface of the wearing article to contact with the wearer's clothes.

### BACKGROUND OF THE INVENTION

The present invention relates to a disposable wearing article provided with tape fasteners. The wearing article includes disposable diapers of pull-on type and open-type having tape fasteners respectively.

Japanese Utility Model Application Publication No. 1994-77719A (Reference 1) discloses a pull-on disposable diaper comprising a liquid-pervious topsheet, a liquid-impervious backsheet and an absorbent pad interposed between these two sheets wherein front and rear waist regions are joined together to form a waist-hole and a pair of leg-holes. In this pull-on diaper of well known art, a waist's intermediate region defined between the waist-hole and the leg-holes is provided with elastic members so that the intermediate region is formed with gathers as the elastic members contract in a waist-surrounding direction. The backsheet is provided on its outer surface with tape fasteners having finger-grips, respectively, so that the respective finger-grips may be oriented in the waist-surrounding direction.

The tape fastener of well known art disclosed in the above-cited Publication is folded in its longitudinal direction in Z- or inverted Z-shape and maintained in this Z- or inverted Z-shape by adhesive applied on segments of a tape tab for the fastener. However, depending on the adhesive force of the adhesive, respective tape segments forming the Z- or inverted Z-shape may be peeled off one from another as the waist's intermediate region is formed with the gathers and obstruct the diaper against being smoothly put on the wearer's body.

### SUMMARY OF THE INVENTION

It is an object of the present invention to provide a wearing article having tape fasteners provided with fastening means improved so that these tape fasteners may be reliably held on the article before the article is actually used and easily peeled off from the article first when the article is actually used.

According to the present invention, there is provided a disposable wearing article having a surface to contact with a wearer's body and a surface to contact with wearer's clothes, the article being contoured by front and rear end zones extending in parallel to each other in a waist-surrounding direction and a pair of opposed side edge zones extending in parallel to each other in a longitudinal direction, the article being composed, in the longitudinal direction orthogonal to the waist-surrounding direction, of a crotch region, a front waist region extending from the crotch region to the front end zone and a rear waist region extending from the crotch region to the rear end zone, the front and rear waist regions being releasably or permanently joined together along the side edge zones so as to form a waist-hole and a pair of leg-holes, the side edge zones of the front or rear waist region being provided with tape fasteners attached thereto so as to extend in the waist-surrounding direction, a tape tab forming each of the tape fasteners having a fixed end zone and a free end zone serving as a finger-grip opposed to each other in a longitudinal direction of the tape tab, the fixed end zone being permanently attached to the surface of the wearing article to contact with the wearer's clothes, the tape tab being adapted to be unfolded from the fixed end zone toward the free end zone as the tape fastener is pulled in the waist-surrounding direction with the free end zone gripped between fingers and, of an inner surface of the tape fastener facing the surface of the wearing article to contact with the wearer's clothes as the tape fastener is unfolded and a surface opposed to the inner surface, the inner surface being at least partially provided with a fastening means attachable to a specified part in any of the inner surface and the surface opposed to the inner surface.

The present invention further comprises the tape material is folded in a Z- or inverted Z-shape so that the tape tab can be unfolded from the fixed end zone toward the free end zone, the tape fastener comprising a first tape segment defining an uppermost layer of the Z- or inverted Z-shape and having the free end zone and a first end zone opposed to the free end zone in the longitudinal direction, the first tape segment being provided, between the both end zones, on the inner surface with a fastening means, a second tape segment lying between the first tape segment and the surface of the wearing article to contact with the wearer's clothes so as to face the surface of the wearing article to contact with the wearer's clothes and having the fixed end zone and a second end zone opposed to the fixed end zone, and a third tape segment interposed between the first tape segment and the second tape segment and connecting the tape segments to each other, the third tape segment having one end zone connected to the first end zone of the first tape segment and the other end zone opposed to the one end zone thereof being connected to the second end zone of the second tape segment and an intermediate zone defined between the one end zone and the other end zone being releasably attached to the first tape segment by means of the fastening means, the one end zone extending in the waist-surrounding direction beyond the fixed end zone of the second tape segment and releasably attached to the surface of the wearing article to contact with the wearer's clothes, and the outer surface of the tape tab folded in any of the Z- or inverted Z-shape is opposed to itself in a non-attached condition in a vicinity of a region in which the second end zone of the second tape segment and the other end zone of the third tape segment are connected to each other.

The present invention includes the following embodiments.

The fastening means is an adhesive coated on the inner surface of the first tape segment. This fastening means can be attached to various materials such as nonwoven fabric and plastic film used in the wearing article.

The fastening means is a hook part provided on the inner surface of a mechanical fastener comprising the hook part and a loop part and the specified part is provided with the loop part. This fastening means is suitable for a use to repeat attaching and releasing of the means on the wearing article.

The loop part is a nonwoven fabric to form any of the surfaces of the wearing article to contact with the wearer's skin or the wearer's clothes. In this embodiment of the present invention there is no need to prepare any specific material for the loop art of the mechanical fastener.

The first end zone of the first tape segment extends in the waist-surrounding direction beyond the one end zone of the third tape segment and is releasably attached to the surface of the wearing article to contact with the wearer's clothes. In a wearing article of this embodiment the tape fastener does not easily peel off from the surface of the wearing article

The one end zone of the third tape segment extends in the waist-surrounding direction beyond the fixed end zone of the second tape segment by a dimension in a range of 1 to 15 mm.

The wearing article may be any of an open-type disposable diaper and a pull-on type disposable diaper. The tape fastener of this embodiment can be used to fit the diaper around the waist of the wearer and also to roll-up the diaper after use.

### BRIEF DESCRIPTION OF THE DRAWINGS

FIG. 1 is a partially cutaway plan view showing a disposable diaper;

FIG. 2 is a sectional view taken along a line II-II in FIG. 1;

FIG. 3 is a view similar to FIG. 2 showing an initial stage of unfolding of the tape fastener;

FIG. 4 is a diagram illustrating a manner in which the tape fasteners are used;

FIG. 5 is a diagram illustrating another manner in which the tape fasteners are used;

FIG. 6 is a view similar to FIG. 2 showing one preferred embodiment of the invention; and

FIG. 7 is a view similar to FIG. 2 showing another preferred embodiment of the invention.

### DETAILED DESCRIPTION OF THE PREFERRED EMBODIMENTS

Details of the disposable wearing article according to the present invention will be more fully understood from the description of a disposable diaper of open-type as a specific embodiment of the invention given hereunder with reference to the accompanying drawings.

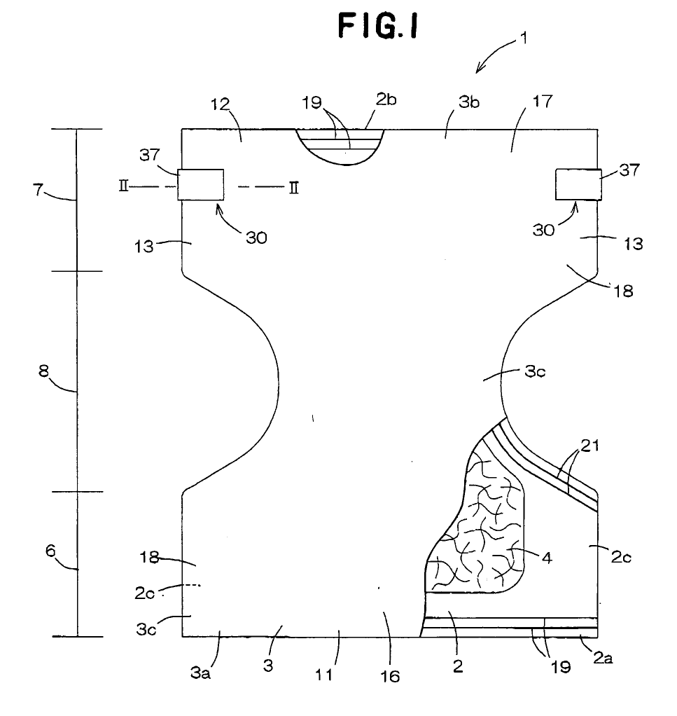

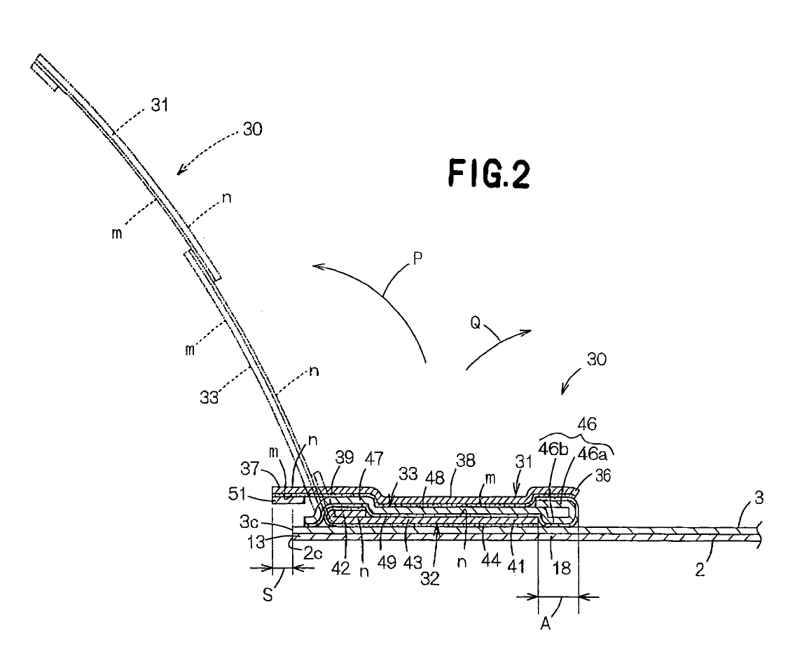

FIG. 1 is a partially cutaway plan view showing a disposable diaper 1 of open-type and FIG. 2 is a sectional view taken along a line II-II in FIG. 1. The diaper 1 basically comprises a liquid-pervious topsheet 2 defining a surface to contact with a wearer's body, a liquid-impervious backsheet 3 to contact with the wearer s clothes and a liquid-absorbent core 4 interposed between these sheets 2, 3. In FIG. 1, the backsheet 3 is illustrated to overlie the topsheet 2. The diaper 1 is contoured by front and rear end zones 11, 12 extending in parallel to each other in a waist-circumferential direction (transverse direction as viewed in FIG. 1) and a pair of side edge zones 13 extending in parallel to each other in a longitudinal direction which is orthogonal to the waist-circumferential direction. In this longitudinal direction, the diaper 1 is composed of a crotch region 8, a front waist region 6 extending between the crotch region 8 and the front end zone 11, and a rear waist region 7 extending between the crotch region 8 and the rear end zone 12. In the crotch region 8, the side edge zones 13 are curved inward. The top- and backsheets 2, 3 extend outward beyond a peripheral edge of the core 4. These portions extending beyond the peripheral edge of the core 4 are overlaid and attached together by means of a hot melt adhesive (not shown) so as to form a front flap 16, a rear flap 17 and side flaps 18. In the front flap 16 and the rear flap 17, waist elastic members 19 are interposed between the top- and backsheets 2, 3 and bonded in a stretched condition to the inner surface of the topsheet 2 and/or the backsheet 3. In the side flaps 18, leg elastic members 21 are interposed between the top- and backsheets 2, 3 along the curved side edge zones 13 in the crotch region 8 and bonded in a stretched condition to the inner surface of the topsheet 2 and/or the backsheet 3. In the rear waist region 7, the respective side flaps 18 have tape fasteners 30 attached to the backsheet 3 in the vicinity of the respective side edge zones 13 These tape fasteners 30 have their lengths extending respectively in the waist-surrounding direction and are folded in a Z- or inverted Z-shape so that the tape fasteners 30 can be unfolded outward beyond the side edge zones 13 of the diaper 1. The top- and backsheets 2, 3 respectively have front and rear end zones 2a, 2b, 3a, 3b defining the front and rear end zones 11, 12 of the diaper 1 and side edge zones 2c, 3c defining the side edge zones 13 of the diaper 1. The topsheet 2 is formed with a liquid pervious nonwoven fabric, a liquid pervious perforated plastic film etc. The backsheet 3 is formed with a liquid impervious nonwoven fabric, a liquid impervious plastic film, a composite from a liquid impervious plastic film and a nonwoven fabric laminated thereon etc.

As will be understood from FIG. 2, each of the tape fastener 30 in a folded state comprises a top tape segment 31 defining a first tape segment, a bottom tape segment 32 defining a second tape segment and an intermediate tape segment 33 defining a third tape segment wherein each of these tape segments 31 through 33 has an inner surface m facing the backsheet 3 when the tape fastener 30 has been unfolded as indicated by imaginary lines and an outer surface n opposed to the surface m. The top tape segment 31 has an inner end zone 36 lying inside the diaper 1 as viewed in the waist-circumferential direction, an outer end zone 37 preferably including a part thereof extending outward beyond the side edge zone 13 so that this outer end zone 37 can be used as a finger-grip and an intermediate zone 38 lying inside the side edge zone 13 wherein the inside surface m of these zones 36 through 38 are coated on the lower surfaces thereof with a second adhesive 39. Referring to FIG. 2, a dimension by which the outer end zone 37 extends outward beyond the side edge zone 13 is indicated by S which is preferably 0.7 mm or longer.

The bottom tape segment 32 lying on the side edge zone 13 and facing the surface of the diaper 1 to contact with the wearer's clothes has an inner end zone 41, an outer end zone 42 and an intermediate zone 43 wherein at least the inner end zone 41 defining the fixed end zone of the tape fastener 30 is permanently attached to the backsheet 3 by means of a first adhesive 44. In the case of the preferred bottom tape segment 32, it will be understood from FIG. 2 that the inner end zone 41, the intermediate zone 43 and the outer end zone 42 except its portion folded back and connected to the intermediate tape segment 33 are permanently attached to the backsheet 3 by means of the first adhesive 44 applied on the inner surfaces m of these zones respectively. The portion of the outer end zone 42 folded back has its surface facing the intermediate tape segment 33 is permanently attached to the intermediate tape segment 33 by means of the first adhesive 44 and its outer surface n facing each other is not attached to itself.

The intermediate tape section 33 also lies inside the side edge zone 13 and has an inner end zone 46, an outer end zone 47 and an intermediate zone 48 wherein respective outer surfaces n of these zones are coated with third adhesive 46.

In such tape fastener 30, the inner end zone 36 of the top tape segment 31 is permanently attached to a portion 46a folded upward from the inner end zone 46 of the intermediate tape segment 33. The outer end zone 37 of the top tape segment 31 is coated with the second adhesive 39 over an appropriate range inclusive of a portion of the outer end zone 37 extending outward in the longitudinal direction of this top tape segment 31 beyond the side edge zone 13 by the dimension S. However, the second adhesive 39 is covered with a film strip 51 and thereby the inner and outer surfaces m, n may become substantially nonadhesive to define the free end zone serving as a finger-grip for the tape fastener 30. The intermediate zone 38 of the top tape segment 31 is releasably attached to the inner surface m of the intermediate tape segment 33 underlying the intermediate zone 38 of the top tape segment 33, i.e. the segment 33 located between the intermediate zone 38 and the bottom tape segment 32.

The distal half 46a folded upward from the inner end zone 46 extending in the waist-surrounding direction of the intermediate tape segment 33 is permanently attached to the top tape segment 31 and the proximal half 46b is releasably attached to the backsheet 3. The proximal half 46b has preferably a length A of 1 to 15 mm as measured in the longitudinal direction of the intermediate tape segment 33. The outer end zone 47 of the intermediate tape segment 33 is permanently attached to the outer end zone 42 of the bottom tape segment 32. The zone of the intermediate tape segment 33 except for the inner and outer end zones 46, 47, i.e., the intermediate zone 48 of the intermediate tape segment 33 is releasably attached to the outer surface n of the bottom tape segment 32 by means of a third adhesive 49. It should be understood that it is possible without departing from the scope of the invention to releasably bond the intermediate zone 48 of the intermediate tape segment 33 to one of the top tape segment 31 and the bottom tape segment 32.

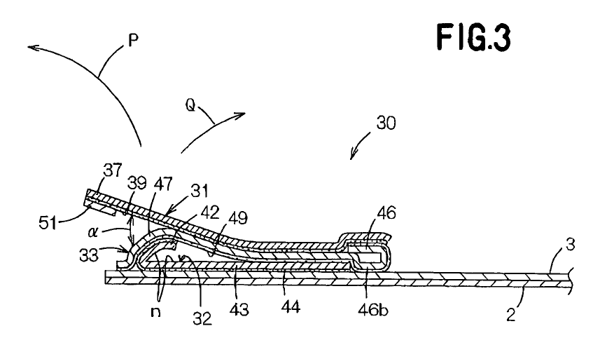

FIG. 3 is a view similar to FIG. 2 showing an initial stage of unfolding of the tape fastener 30. The tape fastener 30 comprises the top tape segment 31, the intermediate tape segment 33 and the bottom tape segment 32 contiguously connected one to another and folded in a Z-shape. Finger-gripping the outer end zone 37 of the top tape segment 31 together with the film strip 51, the tape fastener 30 may be either pulled outwardly in the waist region of the diaper 1 in a direction indicated by an arrow P or pulled inwardly in the waist region of the diaper 1 in a direction indicated by an arrow Q to unfold the tape fastener 30 as indicated by the imaginary lines in FIG. 2. More specifically, it will be understood from FIG. 3 that one of the corners forming the Z-shape at which the bottom tape segment 32 is connected to the intermediate tape segment 33 is lifted up as the tape fastener 30 is pulled in this manner because the outer surface n of the bottom tape segment 32 faces itself in a non-attaching condition in the vicinity of this corner. Consequently, the top tape segment 31 and the intermediate tape segment 33 are spaced from each other at an angle α and so-called a peel force works between these top and intermediate tape segments 31, 33. Under this peel force, these two tape segments 31, 33 are smoothly peeled off from each other. Successively, the proximal half 46b of the inner end zone 46 of the intermediate tape segment 33 is peeled off from the backsheet 3 and an intermediate zone defined between the inner end zone 46 and the outer end zone 47 of the intermediate tape segment 33 is peeled off from the intermediate zone 43 of the bottom tape segment 32. Afterward the tape fastener 30 extends in such a manner as depicted by an imaginary line of FIG. 2. The top tape segment 31 of the tape fastener 30 completely unfolded may be attached by a fastening means, i.e. the adhesive 39, to a specified part of the diaper 1 Such tape fastener 30 can be unfolded merely by pulling the fastener 30 outwardly of the side edge zone 13 and it is not essential to reverse the top tape segment 31 as indicated by the arrow Q in order to unfold the tape fastener 30. From this viewpoint, it is easy to use this tape fastener 30.

In the tape fastener 30, the attaching force of the second adhesive 39 and the third adhesive 49 can be adjusted in various manners so that the second adhesive 39 may be easily peeled off from the intermediate tape segment 33 and the third adhesive 49 may be easily peeled off from the bottom tape segment 32. Such an adjustment can be achieved, for example, by previously selecting the second adhesive 39 and the third adhesive 49 exhibiting the adhesive force lower than that exhibited by the first adhesive 44. It is also possible for such an adjustment to coat the outer surface n of these tape segments 32, 33 with an appropriate release agent such as a silicone oil.

While the tape fastener 30 can be smoothly unfolded, for example, by pulling the tape fastener 30 in the direction indicated by the arrow P when the tape fastener 30 is actually used, the proximal half 46b of the inner end zone 46 of the intermediate tape segment 33 releasably attached to the backsheet 3 neither extends nor peel off unintentionally from the backsheet 3 before an actual use of the tape fastener 30. Even if the outer surface n is entirely coated with a silicone oil, the proximal half 46b of the intermediate tape segment 33 is attached not to the surface n but to the backsheet 3 usually free from coating of a release agent such as a silicone oil. Therefore, it is not likely that the proximal half 46b of the intermediate tape segment 33 might be peeled off from the backsheet 3 unless the tape fastener 30 is pulled in the direction indicated by the arrow P or Q.

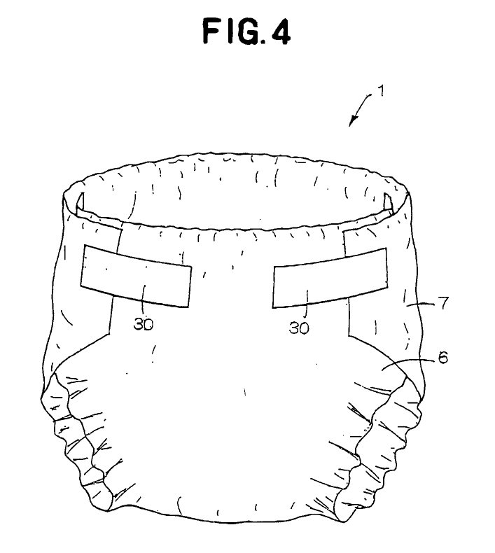

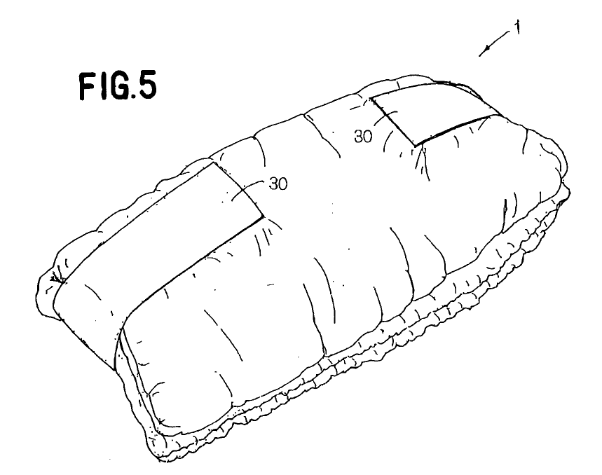

FIGS. 4 and 5 are diagrams illustrating manners in which the tape fasteners 30 are used. In FIG. 4, the diaper 1 is put on the wearer's body and the tape fasteners 30 unfolded outward from the rear waist region 7 and releasably attached to the surface of the front waist region 6 facing the wearer's clothes (not shown) by means of the second adhesive 39. In FIG. 5, the used diaper 1 has been rolled up and is held by the tape fasteners 30 in such a rolled up state. In this way, these tape fasteners 30 are useful not only to put the diaper 1 on the wearer's body but also to hold the used diaper 1 in a rolled up state for disposal.

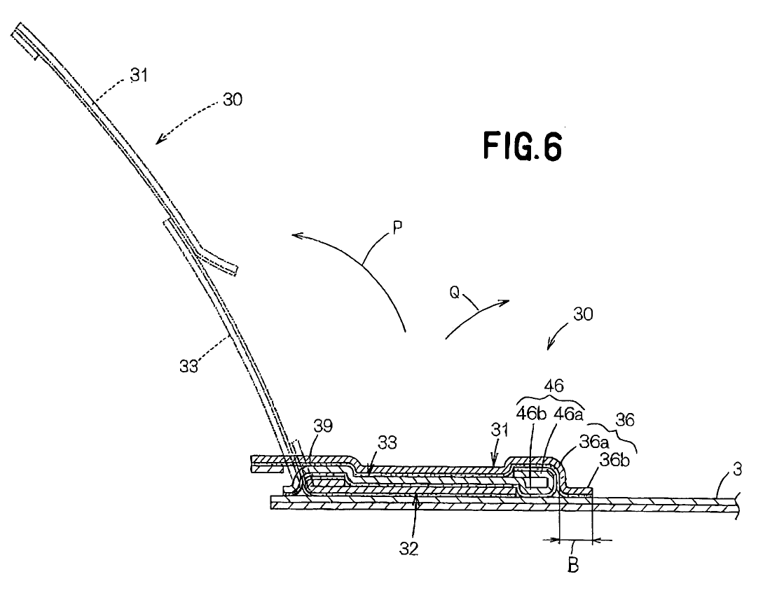

FIG. 6 is a view similar to FIG. 2 showing one preferred embodiment of the invention. In the tape fastener 30 attached to the diaper 1 according to this embodiment, the proximal half 36a partially forming the inner end zone 36 of the top tape segment 31 is permanently attached to the distal half 46a, on one hand, and the distal half 36b extends inwardly of the diaper 1 beyond the inner end zone 46 of the intermediate tape segment 33 and releasably bonded to the backsheet 3 by means of the second adhesive 39, on the other hand. The distal half 36b cooperates with the distal half 46b of the intermediate tape segment 33 to prevent the tape fastener 30 from peeling off from the diaper 1 particularly in its region releasably attached to the backsheet 3. Such preventive effect of the distal half 36b may be further improved by adjusting a dimension B by which the distal half 36b extends inwardly of the diaper 1 to approximately 5 mm. Taking account of both the embodiment shown in FIG. 2 and the embodiment shown in FIG. 6, the dimension B can be specified to be in a range of 0 to 5 mm.

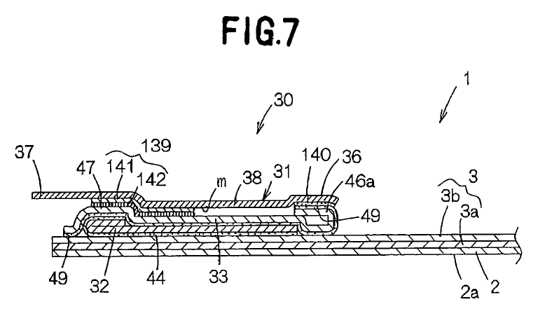

FIG. 7 is a view similar to FIG. 2 showing another preferred embodiment of the present invention. The top tape segment 31 of the tape fastener 30 is formed with a plastic film or a nonwoven fabric. The inner end zone 36 of the top tape segment 31 is coated with a forth adhesive 140 and the inner end zone 46a of the intermediate tape segment 33 is coated with the third adhesive 49. Both of the inner end zones 36, 46a are permanently attached to each other. The inner surface m of the intermediate zone 38 of the top tape segment 31 is provided with a hook part 139 of a mechanical fastener comprising the hook part and a loop part. The hook part 139 has a base sheet 141 and plural hooks 142 extending vertically to the base sheet 141. The base sheet 141 is permanently attached to the inner surface m of the intermediate zone 38. The intermediate tape segment 33 is formed with a nonwoven fabric which has plural loops releasably engageable with the hooks 142. The bottom tape segment 32 is formed with a plastic film or a nonwoven fabric. The bottom tape segment 32 is permanently attached to the backsheet 3 with the first adhesive 44 and also permanently attached to the outer end zone 47 of the intermediate tape segment 33. The topsheet of the diaper 1 is formed with a nonwoven fabric 2a and the backsheet 3 is formed with a composite sheet of a plastic film 3a and a nonwoven fabric 3b laminated on the outer surface of the plastic film 3a. These nonwoven fabrics 2a and 3b have respectively plural loops engageable with the hooks 142. When the outer end 37 of the top tape segment 31 is pulled in the direction indicated by the arrow P of FIG. 1, the tape fastener 30 is deformed as depicted in FIG. 3 and extended straight after the hooks 142 disengage from the loops of the intermediate tape segment 33. The hooks 142 of the tape fastener 30 extended in such a manner can be engaged as a fastening means with the loops provided on any specified parts of the nonwoven fabrics 2a and/or 3b. The diaper 1 having the tape fastener 30 as depicted in FIG. 7 may have the backsheet 3 formed with a plastic film which is provided with a target zone made of a nonwoven fabric engageable with the hooks 142.

While the invention has been described hereinabove with respect to the open-type disposable diaper as a specific embodiment, the invention is applicable also to other disposable wearing articles such as a pull-on type disposable diaper prepared by permanently joining the front and rear waist regions 6, 7 of the diaper 1 together along the respective side edge zones 13 by use of an adhesive or a welding technique, a diaper for incontinent patients or a diaper for training purpose.

The disposable wearing article according to the present invention is characterized in the tape fasteners attached to the article on its surface to contact with the wearer's clothes. Until the article is actually used, each of the tape fasteners is maintained in a folded Z- or an inverted Z-shape. The intermediate tape segment of the tape fastener has its inner end zone extending beyond the inner end zone of the bottom tape segment of the tape fastener and releasably attached to the surface of the article to contact with the wearer's clothes. With such an arrangement, the tape fasteners does not unintentionally peel off from the article. In the vicinity of the region in which the bottom tape segment and the intermediate tape segment are connected to each other, the outer surface of the tape fastener is folded back and facing itself in a non-attaching condition, so the tape fastener can be easily unfolded merely by pulling the outer end zone of the top tape segment of the tape fastener outwardly of the waist region.

What is claimed is:

1. A disposable wearing article having a surface to contact with a wearer's body and a surface to contact with wearer's clothes, said article being contoured by front and rear end zones extending in parallel to each other in a waist-surrounding direction and a pair of opposed side edge zones extending in parallel to each other in a longitudinal direction, said article being composed, in said longitudinal direction orthogonal to said waist-surrounding direction, of a crotch region, a front waist region extending from said crotch region to said front end zone and a rear waist region extending from said crotch region to said rear end zone, said front and rear waist regions being releasably or permanently joined together along said side edge zones so as to form a waist-hole and a pair of leg-holes, said side edge zones of said front or rear waist region being provided with tape fasteners attached thereto so as to extend in said waist-surrounding direction, a tape tab forming each of said tape fasteners having a fixed end zone and a free end zone serving as a finger-grip opposed to each other in a longitudinal direction of said tape tab, said fixed end zone being permanently attached to said surface of said wearing article to contact with the wearer s clothes, said tape fastener being adapted to be unfolded from said fixed end zone toward said free end zone as said tape fastener is pulled in said waist-surrounding direction with said free end zone gripped between fingers and, of an inner surface of said tape fastener facing said surface of said wearing article to contact with the wearer's clothes as said tape fastener is unfolded and a surface opposed to said inner surface, said inner surface being at least partially provided with a fastening means attachable to a specified part in any of the inner surface and the surface opposed to said inner surface, said disposable wearing article further comprising:
said tape material being folded in a Z- or inverted Z-shape so that said tape material can be unfolded from said fixed end zone toward said free end zone, said tape fastener comprising a first tape segment defining an uppermost layer of said Z- or inverted Z-shape and having said free end zone and a first end zone opposed to said free end zone in said longitudinal direction, said first tape segment being provided, between said both end zones, on said inner surface with said fastening means, a second tape segment lying between said first tape segment and said surface of said wearing article to contact with the wearer's clothes so as to face said surface of said wearing article to contact with said wearer's clothes and having said fixed end zone and a second end zone opposed to said fixed end zone, and a third tape segment interposed between said first tape segment and said second tape segment and connecting said tape segments to each other, said third tape segment having one end zone connected to said first end zone of said first tape segment and the other end zone opposed to said one end zone thereof being connected to said second end zone of said second tape segment and an intermediate zone defined between said one end zone and said other end zone being releasably attached to said first tape segment by means of said fastening means adhesive, said one end zone extending in said waist-surrounding direction beyond said fixed end zone of said second tape segment and releasably attached to said surface of said wearing article to contact with said wearer's clothes, and said outer surface of said tape tab folded in any of said Z- or inverted Z-shape is opposed to itself in a non-attached condition in a vicinity of a region in which said second end zone of said second tape segment and said other end zone of said third tape segment are connected to each other.

2. The disposable wearing article according to claim 1, wherein said fastening means is an adhesive coated on said inner surface of said first tape segment.

3. The disposable wearing article according to claim 1, wherein said fastening means is a hook part provided on said inner surface of a mechanical fastener comprising said hook part and a loop part and said specified part is provided with said loop part.

4. The disposable wearing article according to claim 3, wherein said loop part is a nonwoven fabric to form any of said surfaces to contact with said wearer's skin or said wearer's clothes.

5. The disposable wearing article according to claim 1, wherein said first end zone of said first tape segment extends in said waist-surrounding direction beyond said one end zone of said third tape segment and is releasably attached to said surface of said wearing article to contact with said wearer's clothes.

6. The disposable wearing article according to claim 1, wherein said one end zone of said third tape segment extends in said waist-surrounding direction beyond said fixed end zone of said second tape segment by a dimension in a range of 1 to 15 mm.

7. The disposable wearing article according to claim 1, wherein said wearing article is any of an open-type disposable diaper and a pull-on type disposable diaper.
